(19) United States
(12) Patent Application Publication
(10) Pub. No.: US 2008/0177353 A1
(43) Pub. Date: Jul. 24, 2008

(54) Cochlear implant device, extracorporeal sound collector, and cochlear implant system having the same

(76) Inventors: Takashi Hirota, Uzi (JP); Mayumi Yamaguchi, Atsugi (JP)

Correspondence Address: ERIC ROBINSON, PMB 955, 21010 SOUTHBANK ST., POTOMAC FALLS, VA, 20165, US

(21) Appl. No.: 12/004,512

(22) Filed: Dec. 21, 2007

(30) Foreign Application Priority Data
Dec. 28, 2006 (JP) 2006-354767

(51) Int. Cl. A61N 1/00; A61F 2/18

(52) U.S. Cl. 607/57; 623/10; 607/56

(57) ABSTRACT

An object is to provide a cochlear implant system (also known as an artificial inner ear system) which is easy to use with little interference with daily activities. A cochlear implant device (or artificial inner ear device) includes an inner ear electrode, an information processing circuit, a transmitter/receiver circuit, a charging circuit, and a battery; and the battery is charged with electromagnetic waves received by the transmitter/receiver circuit through the charging circuit. In addition, the power stored in the battery is supplied to the cochlear implant device. Further, the electromagnetic waves received by the transmitter/receiver circuit are converted into a signal by the information processing circuit, and the signal is provided from the inner ear electrode to stimulate the auditory nerve.

### BACKGROUND OF THE INVENTION

1. Field of the Invention

The present invention relates to a cochlear implant device, an extracorporeal sound collector, and a cochlear implant system having each of them.

2. Description of the Related Art

A cochlear implant system is a device by which an electrical signal is directly applied to an inner ear (a cochlea) to make a brain perceive sound. The cochlear implant system has a structure formed of two main parts: a speech processor (referred to as an extracorporeal sound collector in this specification) and an inner ear electrode (referred to as a cochlear implant device in this specification). The speech processor (extracorporeal sound collector) converts a detected external sound into an electrical signal and transmits it to the inner ear electrode (cochlear implant device). The inner ear electrode (cochlear implant device) which receives the electrical signal is to provide a stimulus from an electrode inserted inside a cochlea to an auditory nerve. By use of such a cochlear implant system, a hearing impairment where a conventional hearing aid has not been supplied can be improved (Patent Document 1: Japanese Published Patent Application No. 2006-204646 and Patent Document 2: Japanese Translation of PCT International Application No. 2004-527194).

A cochlear implant device performs wireless communication by an electromagnetic induction method from an extracorporeal sound collector and receives a supply of power. Accordingly, the cochlear implant device does not have a power source such as a cell. Specifically, a coil antenna of an extracorporeal sound collector is arranged so as to be coupled to a coil antenna of a cochlear implant through skin by electromagnetic coupling. The antenna portion of the extracorporeal sound collector is referred to as a headpiece and is a circle having a diameter of about 3 cm, a thickness of about 8 mm, and a weight of about 5 g. This headpiece is used by being attached with a magnet so as to be opposed to the coil antenna of the cochlear implant that is embedded in a scalp behind an ear with skin in between the headpiece and the coil antenna.

The extracorporeal sound collector includes the headpiece, a sound collecting microphone, a signal processor, and the like and operates with a cell as a power source. In the case of one type of extracorporeal sound collector in which a sound collecting microphone and a signal processor are separated from each other, the signal processor is used by being placed in a breast pocket or fixed to a belt, and the sound collecting microphone is used by being worn on an ear. The weight of the sound collecting microphone is about 5 g to 10 g. Meanwhile, in the case of another type of extracorporeal sound collector in which a sound collecting microphone and a signal processor are formed integrally, the extracorporeal sound collector is used by being worn on an ear or fixed to a belt or the like so as to be exposed to external. For example, in the case where an extracorporeal sound collector is used by being worn on an ear, weight placed on the ear is about 12 g.

However, there are some major problems with the cochlear implant system in the wearing of a headpiece. For example, one problem is with how the headpiece feels while it is being used. In the case of wearing a headpiece, the strength of a magnet that is used for attachment is limited. Although some of the hair over which the headpiece is attached need not be shaved off, when the headpiece is placed over the hair, the headpiece is unstable depending on the amount of hair. Therefore, the headpiece cannot be worn properly depending on the hairstyle and the thickness of the skin. Furthermore, there is a case in which unnatural discomfort occurs due to attachment while the headpiece is worn and a case in which a hairstyle cannot be chosen freely.

In addition, a speech processor which is used by being worn on an ear may be broken because of moisture from sweat, hair, dust, or the like, in some cases.

A speech processor which is used by being worn on the ear is integrally formed with a sound collecting microphone and a signal processor, and the speech processor can be used for from 60 hours to 80 hours with one battery change. However, because such a speech processor has a relatively high output and needs to be small in size and lightweight, a zinc-air cell used exclusively by the speech processor is required to be used. This dedicated cell is disposable and incurs maintenance costs while being used. Furthermore, the range for temperature and humidity in which the dedicated cell can be used is narrow, and the dedicated cell cannot be used at a high temperature, at a low temperature, in high humidity, or in a dry state.

In the case where a speech processor whose signal processor is placed in a breast pocket and whose sound collecting microphone is worn on an ear is used, a headpiece, the sound collecting microphone, and the signal processor are connected to one another with a cable. This cable disturbs operations of a user, and the cable may be cut so that the speech processor is broken in some cases. For this reason, a user often carries a spare cable.

With the above wearing method, because the speech processor (extracorporeal sound collector) needs to be removed when a user enters water, such as when bathing or swimming, a cochlear implant system cannot be used.

### SUMMARY OF THE INVENTION

In view of the foregoing problems, an object of the present invention is to provide a cochlear implant system which is easy to use with little interference with daily activities.

One feature of the present invention is a cochlear implant device including an inner ear electrode, an information processing circuit, a transmitter/receiver circuit, a charging circuit, and a battery, and the battery is charged with electromagnetic waves received by the transmitter/receiver circuit through the charging circuit. In addition, the power stored in the battery is supplied to the cochlear implant device. Further, the electromagnetic waves received by the transmitter/receiver circuit are converted into a signal by the information processing circuit, and the signal is provided from the inner ear electrode to stimulate the auditory nerve.

Another feature of the present invention is an extracorporeal sound collector including a microphone, an external input circuit, an information processing circuit, a transmitter/receiver circuit, a charging circuit, and a battery, and sounds detected by the microphone are converted into a signal by the information processing circuit, the signal is transmitted by the transmitter/receiver circuit to a cochlear implant device, along with electromagnetic waves of power with which the battery is charged through the transmitter/receiver circuit being transmitted to the cochlear implant device.

Another feature of the present invention is a cochlear implant system including a cochlear implant device having an inner ear electrode, a first information processing circuit, a first transmitter/receiver circuit, a first charging circuit, and a first battery as well as an extracorporeal sound collector having a microphone, an external input circuit, a second information processing circuit, a second transmitter/receiver circuit, a second charging circuit, and a second battery. In the first transmitter/receiver circuit and the second transmitter/receiver circuit, signals related to sounds detected by the microphone are transmitted and received, along with power stored in the second battery being supplied to the first battery by use of electromagnetic waves.

Here, the above first information processing circuit includes an amplifier circuit, a central arithmetic processing circuit, and the like. In addition, the above second information processing circuit includes an external input circuit, an amplifier circuit, a central arithmetic processing circuit, and the like.

Here, the first transmitter/receiver circuit that is provided in the cochlear implant device and the second transmitter/receiver circuit that is provided in the extracorporeal sound collector each include at least one antenna, a capacitor, a demodulation circuit, a decoding circuit, a logic operation/control circuit, a memory circuit, an encoding circuit, and a modulation circuit.

The first charging circuit that is provided in the cochlear implant device includes a rectifier circuit which rectifies an induced electromotive force that is generated in the antenna which is included in the first transmitter/receiver circuit that is provided in the cochlear implant, a current/voltage control circuit, and a charge control circuit. The second charging circuit that is provided in the extracorporeal sound collector includes a rectifier circuit which rectifies power inputted from an external power source, a current/voltage control circuit, and a charge control circuit.

In the cochlear implant system of the present invention, the inner ear electrode is connected to the first amplifier circuit that is provided in the cochlear implant device, and the first amplifier circuit is connected to the first central arithmetic processing circuit that is provided in the cochlear implant device. In addition, the first transmitter/receiver circuit that is provided in the cochlear implant device is connected to the first central arithmetic processing circuit that is provided in the cochlear implant device and the first charging circuit that is provided in the cochlear implant device, and the first charging circuit that is provided in the cochlear implant device is connected to the first battery that is provided in the cochlear implant device. Further, the first battery that is provided in the cochlear implant device supplies power to the cochlear implant device.

The microphone that is included in the extracorporeal sound collector is connected to the external input circuit, and the external input circuit is connected to the second amplifier circuit that is provided in the extracorporeal sound collector. However, the extracorporeal sound collector may have a structure in which the microphone is connected to an amplifier circuit without any external input circuit being provided. In addition, the second amplifier circuit that is provided in the extracorporeal sound collector is connected to the second central arithmetic processing circuit that is provided in the extracorporeal sound collector, and the second transmitter/receiver circuit that is provided in the extracorporeal sound collector is connected to the second central arithmetic processing circuit that is provided in the extracorporeal sound collector and the second charging circuit that is provided in the extracorporeal sound collector. Further, the second charging circuit that is provided in the extracorporeal sound collector is connected to the second battery that is provided in the extracorporeal sound collector, and the second battery that is provided in the extracorporeal sound collector supplies power to the extracorporeal sound collector.

Here, the second battery that is provided in the extracorporeal sound collector is charged using the external power source through the second charging circuit that is provided in the extracorporeal sound collector. In addition, as a method of charging of the first battery that is provided in the cochlear implant device, electromagnetic waves transmitted from the second transmitter/receiver circuit that is provided in the extracorporeal sound collector are received by the first transmitter/receiver circuit that is provided in the cochlear implant device, and the first battery is charged through the first charge control circuit that is provided in the cochlear implant device.

As described above, the cochlear implant device of the present invention includes a battery which is a self-driving power source that is not originally included in the device. Furthermore, a method of communication with the extracorporeal sound collector is not limited to being an electromagnetic coupling method, and a communication distance with the extracorporeal sound collector can be extended when the cochlear implant device has a structure in which communication is performed by use of electromagnetic waves. Accordingly, a user of a cochlear implant system can use an extracorporeal sound collector at a place other than one's head and be released from the difficulty in wearing a headpiece on one's head. As a result of this, the daily life of a user of a cochlear implant system can be improved.

### BRIEF DESCRIPTION OF THE DRAWINGS

FIG. 1 is a diagram showing a structure of the present invention in which a cochlear implant system includes a cochlear implant device and an extracorporeal sound collector.

FIG. 2 is a diagram showing one mode of the present invention in which a cochlear implant system is used and a cochlear implant device and an extracorporeal sound collector are worn.

FIGS. 3A and 3B are diagrams showing a mode in which a cochlear implant system of the present invention is used.

FIG. 4 is a diagram showing another structure of an extracorporeal sound collector of the present invention.

FIGS. 5A and 5B are diagrams each showing a part of a cochlear implant device of the present invention.

FIGS. 6A to 6D are diagrams showing a manufacturing process of a cochlear implant device of the present invention.

FIGS. 7A and 7B are diagrams showing a manufacturing process of a cochlear implant device of the present invention.

FIGS. 8A and 8B are diagrams showing a manufacturing process of a cochlear implant device of the present invention.

FIGS. 9A and 9B are diagrams which showing a manufacturing process of a cochlear implant device of the present invention.

FIGS. 10A and 10B are diagrams showing a manufacturing process of a cochlear implant device of the present invention.

### DETAILED DESCRIPTION OF THE INVENTION

Hereinafter, embodiment modes of the present invention will be described with reference to the accompanying drawings. However, the present invention is not limited to the following description. As can be easily understood by those skilled in the art, the modes and details of the present invention can be changed in various ways without departing from the spirit and scope of the present invention. Thus, the present invention should not be interpreted as being limited to the following description of the embodiment modes. Note that the same reference numerals are commonly used to denote the same components among different drawings in structures of the present invention explained below.

### Embodiment Mode 1

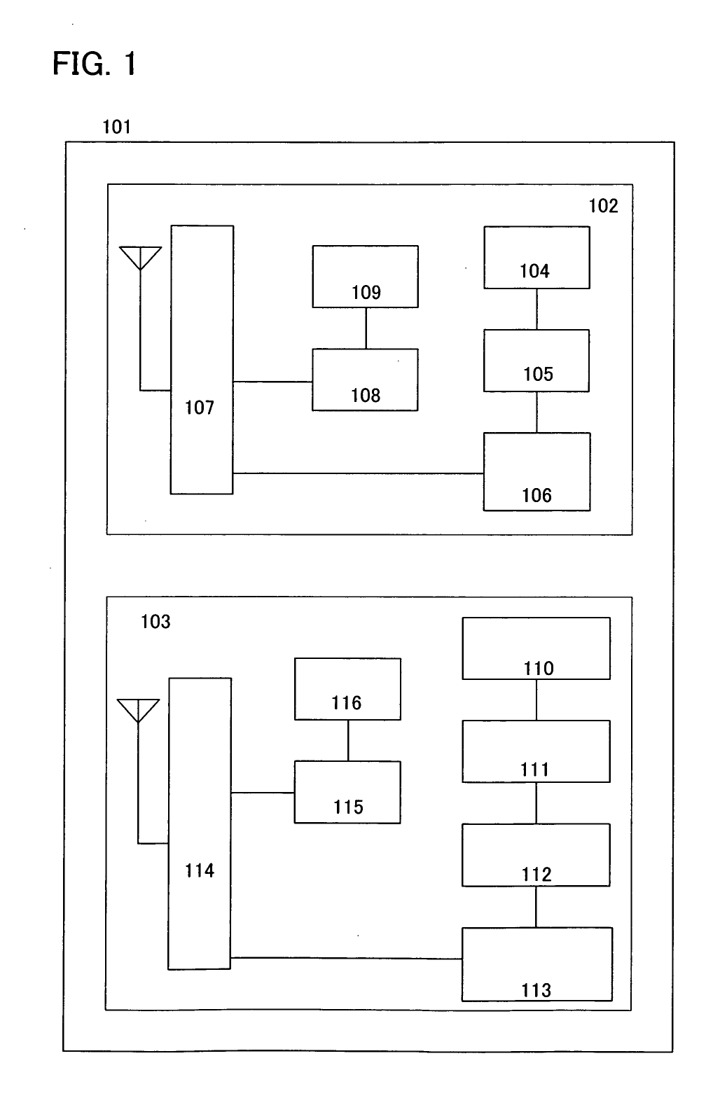

In this embodiment mode of the present invention, a cochlear implant device, an extracorporeal sound collector, and a cochlear implant system having each of them will be described. A cochlear implant system 101 of the present invention includes a cochlear implant device 102 which is embedded in a body and transmits information for sounds to an auditory nerve, and an extracorporeal sound collector 103 which detects ambient sounds from outside the body and transmits them to the cochlear implant device (see FIG. 1).

First, the cochlear implant device 102 will be described. The cochlear implant device 102 of the cochlear implant system 101 includes an inner ear electrode 104, an amplifier circuit 105, a central arithmetic processing circuit 106, a transmitter/receiver circuit 107, a charging circuit 108, and a battery 109.

The inner ear electrode 104 provides electric stimulation to the auditory nerve of an inner ear. The amplifier circuit 105 amplifies a signal that is to be transmitted to the inner ear electrode 104. The central arithmetic processing circuit 106 performs information processing in order to communicate with the extracorporeal sound collector 103. The transmitter/receiver circuit 107 performs wireless communication with the extracorporeal sound collector 103. The charging circuit 108 charges the battery with electromagnetic waves from the extracorporeal sound collector 103 as power. The battery supplies power to the inner ear electrode 104, the amplifier circuit 105, the central arithmetic processing circuit 106, the transmitter/receiver circuit 107, the charging circuit 108, and the like of the cochlear implant device 102.

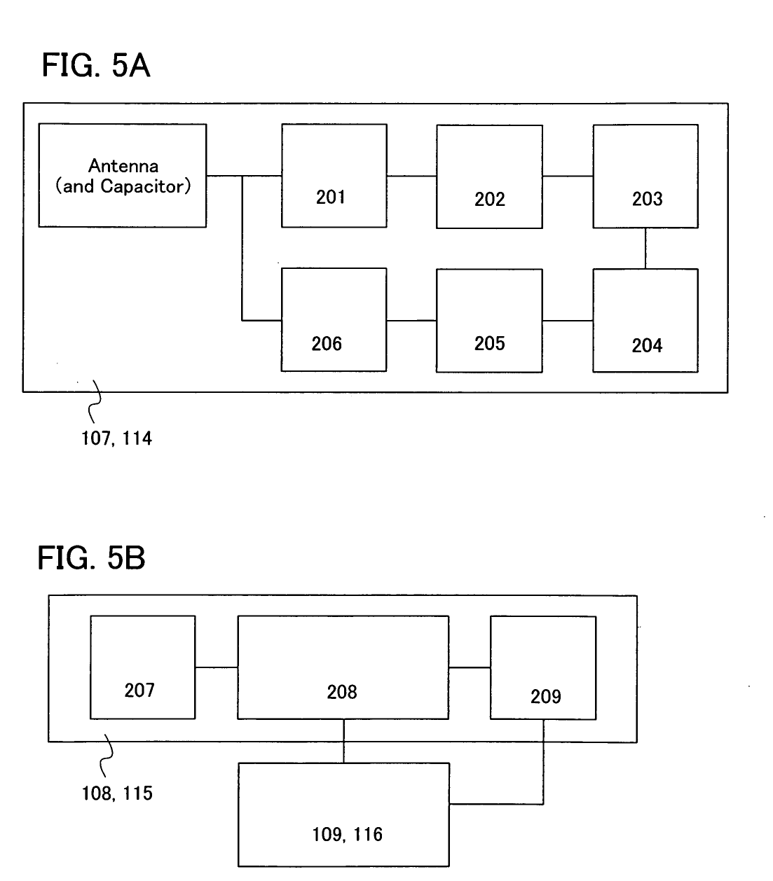

Here, the transmitter/receiver circuit 107 that is provided in the cochlear implant device is a circuit which performs wireless communication with the extracorporeal sound collector 103, as shown in FIG. 5A. Therefore, for example, the transmitter/receiver circuit 107 includes at least one antenna, a capacitor, a demodulation circuit 201, a decoding circuit 202, a logic operation/control circuit 203, a memory circuit 204, an encoding circuit 205, and a modulation circuit 206. By using such a structure, the demodulation circuit 201 demodulates and extracts data included in an induced voltage generated in the antenna, and the data is decoded by the decoding circuit 202. Then, data processed by the logic operation/control circuit 203 or the like is made to be an encoded signal by the encoding circuit 205, and a carrier wave is modulated by the modulation circuit 206 based on the encoded signal.

The charging circuit 108 that is provided in the cochlear implant device includes a rectifier circuit 207 which rectifies an induced electromotive force generated in the antenna, a current/voltage control circuit (also referred to as a regulator) 208, and a charge control circuit 209, as shown in FIG. 5B. Specifically, an AC induced electromotive voltage is generated when the antenna that is included in the transmitter/receiver circuit 107 which is provided in the cochlear implant device receives electromagnetic waves, and the AC induced electromotive voltage is inputted to a dielectric circuit. The inputted AC induced electromotive voltage is rectified by the rectifier circuit 207 and controlled by the current/voltage control circuit 208 so as to be a voltage suitable for charging to charge the battery 109. At this time, the charge control circuit 209 monitors the state of charging of the battery 109. For example, the charge control circuit 209 monitors the voltage of the battery 109; when the voltage of the battery 109 is equal to or exceeds a given value, the charge control circuit 209 stops the current/voltage control circuit 208 or the like, and charge is terminated by cutting the electrical connection between the current/voltage control circuit 208 and the battery 109.

The battery 109 charged in this manner supplies power to each circuit, such as the inner ear electrode 104, the amplifier circuit 105, the central arithmetic processing circuit 106, the transmitter/receiver circuit 107, and the charging circuit 108, in order to drive the entire cochlear implant device 102. In this way, the cochlear implant device 102 including a wireless communication function includes a battery which is a self-driving power source that is not originally included in the device. Furthermore, a method of communication with the extracorporeal sound collector is not limited to being an electromagnetic coupling method, and a communication distance of wireless communication can be extended when the cochlear implant device has a structure in which communication is performed by use of electromagnetic waves.

The amplifier circuit 105, the central arithmetic processing circuit 106, the transmitter/receiver circuit 107, and the charging circuit 108 of the cochlear implant device 102 may each be formed of a field effect transistor (FET) or a thin film transistor by use of a single crystal silicon substrate or an SOI substrate. Alternatively, a given circuit may be formed of a combination of a field effect transistor and a thin film transistor. When thin film transistors are used for the above circuits, the cochlear implant device can be made thinly.

Next, the extracorporeal sound collector 103 will be described. The extracorporeal sound collector 103 of the cochlear implant system 101 includes a microphone 110, an external input circuit 111, an amplifier circuit 112, a central arithmetic processing circuit 113, a transmitter/receiver circuit 114, a charging circuit 115, and a battery 116. The microphone 110 detects external sounds. A signal from the microphone 110 or from another external device is inputted to the external input circuit 111. However, a structure may be used in which the extracorporeal sound collector 103 does not include the external input circuit 111 and the microphone 110 is connected to the amplifier circuit 112, as well. The amplifier circuit 112 amplifies an analog audio signal that is inputted from the microphone 110 or the like. The central arithmetic processing circuit 113 decomposes the audio signal that is amplified by the amplifier circuit 112 into each frequency and changes it into an electric signal that is to be used by the inner ear electrode 104 of the cochlear implant device 102. The transmitter/receiver circuit 114 performs wireless communication with the cochlear implant device 102. The charging circuit 115 supplies power supplied from a cell or from an external power source to the battery 116, and the battery 116 supplies power to the extracorporeal sound collector 103.

Here, the transmitter/receiver circuit 114 can have a structure that is almost the same as that of the transmitter/receiver circuit 107 that is provided in the cochlear implant, as shown in FIG. 5A. Specifically, the transmitter/receiver circuit 114 includes an oscillator circuit which oscillates electromagnetic waves, as well as at least one antenna, a capacitor, a demodulation circuit, a decoding circuit, a logic operation/control circuit, a memory circuit, an encoding circuit, and a modulation circuit. The charging circuit 115 includes the rectifier circuit 207, the current/voltage control circuit 208, and the charge control circuit 209, and the like to supply power that is supplied from a cell or from an external power source to the battery 116 that is provided in the cochlear implant as shown in FIG. 5B, and the battery is charged from the external power source through the charging circuit 115. The battery 116 that is charged in this way supplies power to each circuit so as to drive the entire extracorporeal sound collector 103. Here, the extracorporeal sound collector 103 can have not a structure that includes the charging circuit 115 and the battery 116 that is charged by the charging circuit 115 but a structure that includes a general cell.

The external input circuit 111, the amplifier circuit 112, the central arithmetic processing circuit 113, the transmitter/receiver circuit 114, and the charging circuit 115 of the extracorporeal sound collector 103 may each be formed of a field effect transistor (FET) or a thin film transistor by use of a single crystal silicon substrate or an SOI substrate. Alternatively, a given circuit may be formed of a combination of a field effect transistor and a thin film transistor. The microphone 110 may be formed using a MEMS device. When a MEMS device is used for the microphone 110, a weak signal can also be detected; therefore, the microphone is small and high sensitivity, and the microphone can detect a weak sound.

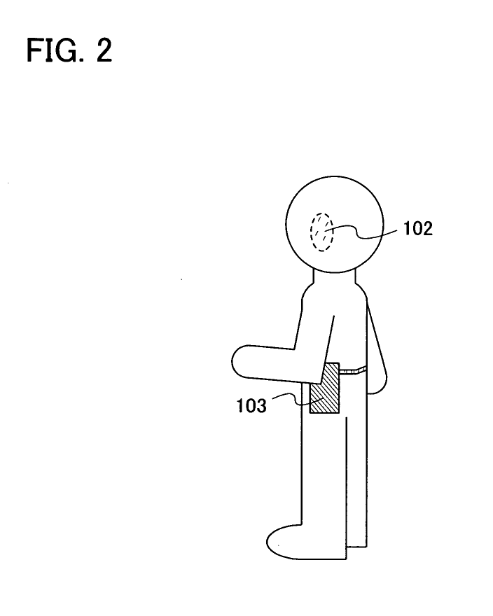

Next, a usage mode of the cochlear implant system 101 of the present invention will be described. As shown in FIG. 2, the cochlear implant device 102 is embedded into a body, and the extracorporeal sound collector 103 is fixed to a belt or placed in a pocket. In FIG. 2, an example is shown in which the extracorporeal sound collector 103 is fixed to a belt.

Note that the extracorporeal sound collector 103 is desirably fixed so that the microphone is exposed in order that external sounds can be detected with high accuracy.

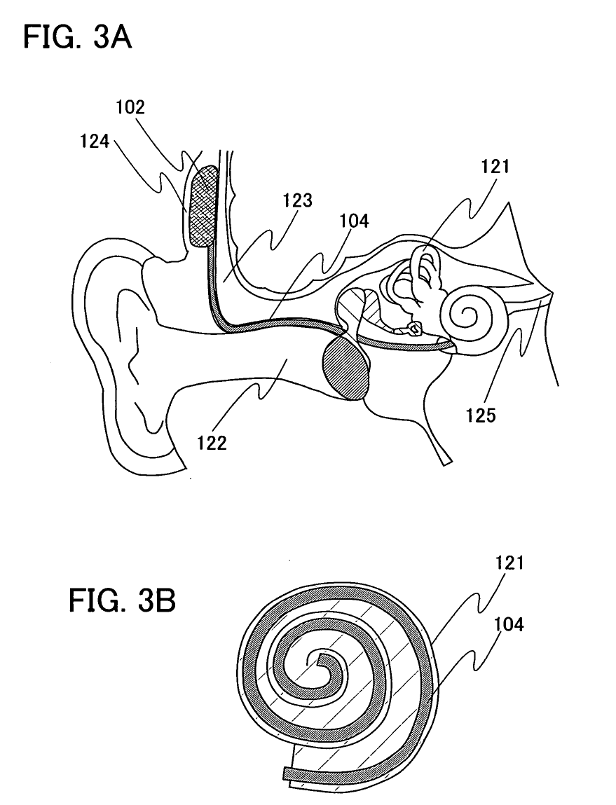

FIGS. 3A and 3B are diagrams showing a cross section of an ear in order to show the arrangement of the cochlear implant device 102.

The cochlear implant device 102 is embedded between an external auditory canal 122 and a skull 123 and between skin 124 and the skull 123 (see FIG. 3A). FIG. 3B shows a cross-sectional view of a cochlea. The inner ear electrode 104 is inserted into a cochlea 121 and is connected to an auditory nerve. Since wireless communication is performed by use of electromagnetic waves, a neck or a back can be provided with components other than the inner ear electrode 104 of the cochlear implant device 102. Furthermore, each circuit can be dispersed and embedded in the body in consideration of the function of each circuit in such a way that the amplifier circuit 105, the central arithmetic processing circuit 106, the charging circuit 108, and the battery 109 are embedded together in one portion, such as in the external auditory canal 122, and just the transmitter/receiver circuit 107 and the antenna are embedded in the neck, or the like.

The cochlear implant system 101 provided in this way functions as described hereinafter. First, external sounds are detected by the microphone 110 that is provided in the extracorporeal sound collector. Then, information for the external sounds is amplified by the amplifier circuit 112 through the external input circuit 111; analog-to-digital conversion is performed; and decomposition is performed into each frequency to be processed by the central arithmetic processing circuit 113 into a signal required by the cochlear implant device 102. Then, a signal is transmitted from the transmitter/receiver circuit 114 to the cochlear implant device 102.

Next, in the cochlear implant device 102, a signal transmitted from the extracorporeal sound collector 103 is received by the transmitter/receiver circuit 107. Then, signal processing is performed by the central arithmetic processing circuit 106, a signal is amplified by the amplifier circuit 105, and an auditory nerve 125 is stimulated by the inner ear electrode 104. Accordingly, a user of the cochlear implant device can perceive sounds detected by the microphone.

In addition, a function related to a supply of power of the cochlear implant system 101 of the present invention is as described hereinafter. First, in the extracorporeal sound collector 103, power is supplied from a cell or from an external power source to the charging circuit 115, and the charging circuit charges the battery 116. The charged battery 116 supplies power to each circuit of the extracorporeal sound collector 103 so as to drive the extracorporeal sound collector 103, along with the charged battery 116 supplying power to the transmitter/receiver circuit 114 so as to supply power to the cochlear implant device 102. The transmitter/receiver circuit 114 that is provided in the extracorporeal sound collector transmits electromagnetic waves in order to supply power to the cochlear implant device 102.

Next, in the transmitter/receiver circuit 107 that is provided in the cochlear implant device, electromagnetic waves transmitted from the extracorporeal sound collector 103 are received, the power is rectified by the charging circuit 108, and the battery 109 is charged. Then, the charged battery 109 supplies power to each circuit of the cochlear implant device 102 so as to drive the cochlear implant device 102.

Note that the cochlear implant device 102 can be charged wirelessly from the extracorporeal sound collector 103 as described above; however, the cochlear implant device 102 can have a structure where it can be charged by a wireless charging device built into an article for daily life such as a pillow, a bed, a hat, or furniture.

The cochlear implant device 102 of the present invention includes a battery which is a self-driving power source that is not originally included in the device. Furthermore, a method of communication with the extracorporeal sound collector is not limited to being an electromagnetic coupling method, and a communication distance can be extended when the cochlear implant device has a structure in which communication is performed by use of electromagnetic waves. Therefore, even when a distance between the extracorporeal sound collector 103 and the cochlear implant device 102 increases to some extent, sounds can be heard.

Furthermore, a headpiece need not be mounted on the head, worn on the ear, or the like, and a user can be released from discomfort or difficulty in wearing the extracorporeal sound collector 103, in particular, a transmitter/receiver portion (headpiece), in the vicinity of an ear.

The cochlear implant device 102 of the present invention has a structure with a battery which can be charged wirelessly. The cochlear implant device 102 and the extracorporeal sound collector 103 are made to be waterproof, by which swimming and bathing while the extracorporeal sound collector 103 is being worn can be enabled.

### Embodiment Mode 2

In this embodiment mode, an example is shown in which the cochlear implant system 101 is used by use of a function included in the extracorporeal sound collector 103 of the present invention.

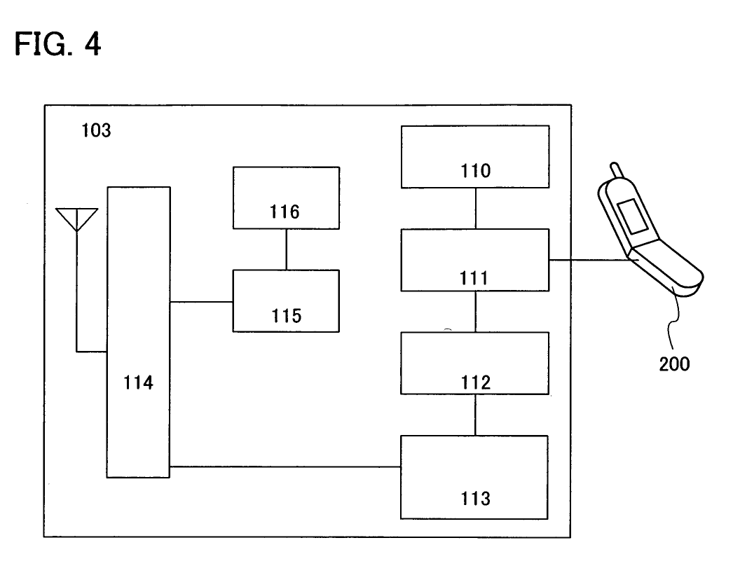

The extracorporeal sound collector 103 of the present invention includes the external input circuit 111. A radio, a cellular phone 200, a music player, or the like is connected to this external input circuit 111 so that a user of the cochlear implant system 101 can hear sounds outputted from the connected device (see FIG. 4).

For example, when information for sounds input from the outside is an analog signal, a structure can be used in which the external input circuit 111 is provided between the microphone 110 and the amplifier circuit 112. When information for sounds is input by a digital signal, a structure can be used in which the external input circuit 111 and the central arithmetic processing circuit 113 are connected to each other. Needless to say, a structure corresponding to an input of either an analog signal or a digital signal can also be used.

In this manner, even if a person is hard-of-hearing, he or she can enjoy entertainment such as music or radio or can communicate with another person by cellular phone by use of the cochlear implant system 101 of the present invention.

Note that this embodiment mode can be freely combined with the above embodiment mode.

### Embodiment Mode 3

In this embodiment mode, an example of a method for manufacturing the cochlear implant device described in Embodiment Modes 1 and 2 will be described with reference to FIGS. 1, 6A to 6D, 7A and 7B, 8A and 8B, 9A and 9B, and 10A and 10B. Although the cochlear implant device can be formed of a field effect transistor by use of a semiconductor substrate or an SOI substrate, a structure in which an antenna, a charging circuit, and a transmitter/receiver circuit are provided over the same substrate will be described in this embodiment mode. In addition, an example of a method for manufacturing a charging circuit and a transmitter/receiver circuit by use of a thin film transistor will be described. Note that an antenna, a charging circuit, a transmitter/receiver circuit, a central arithmetic processing circuit, an amplifier circuit, and the like can be formed over a substrate and thin film transistors as transistors included in the antenna, the charging circuit, the transmitter/receiver circuit, the central arithmetic processing circuit, the amplifier circuit, and the like can be made so that miniaturization can be achieved, which is preferable.

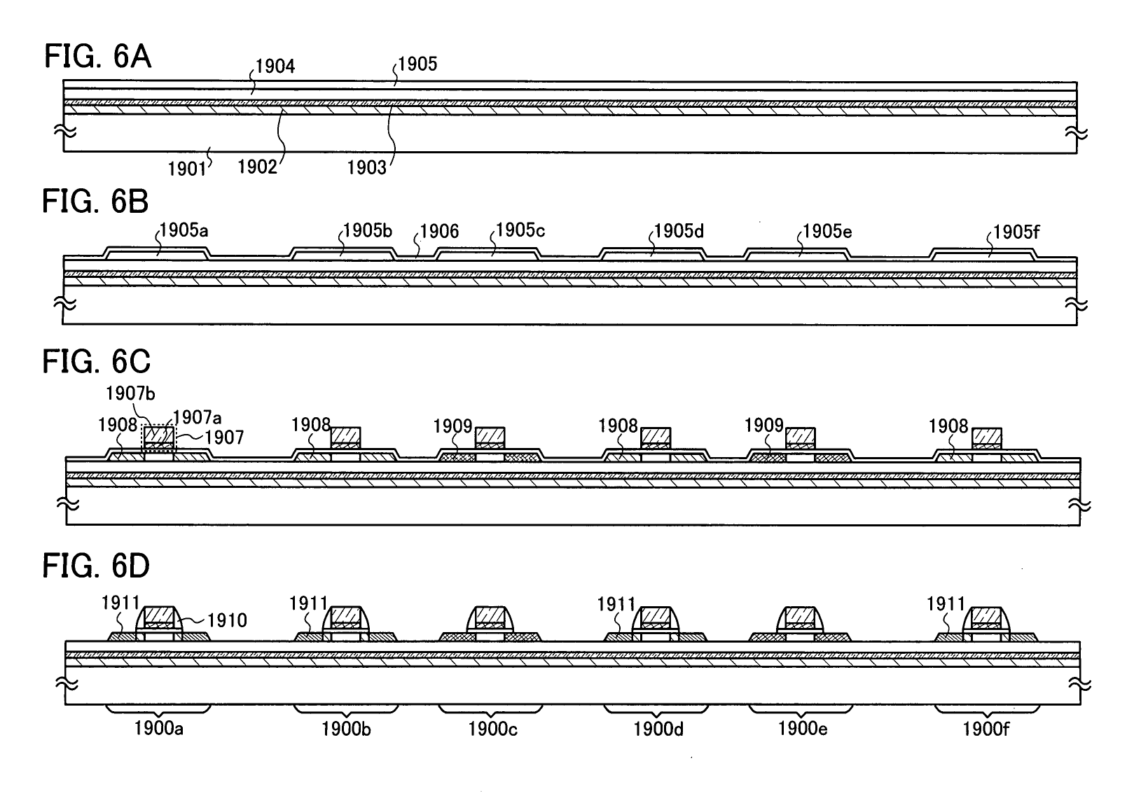

First, as shown in FIG. 6A, a separation layer 1903 is formed over a surface of a substrate 1901 with an insulating film 1902 interposed therebetween. Next, an insulating film 1904, which serves as a base film, and a semiconductor film 1905 (e.g., a film which includes amorphous silicon) are stacked. Note that the insulating film 1902, the separation layer 1903, the insulating film 1904, and the semiconductor film 1905 can be formed in succession.

Further, the substrate 1901 may be a glass substrate, a quartz substrate, a metal substrate (e.g., a stainless steel substrate or the like), a ceramic substrate, or a semiconductor substrate, such as a Si substrate. Alternatively, a plastic substrate formed of polyethylene terephthalate (PET), polyether sulfone (PES), acrylic, or the like can be used. Note that in this step, the separation layer 1903 is provided over an entire surface of the substrate 1901 with the insulating film 1902 interposed therebetween; however, if necessary, the separation layer may be selectively provided by use of a photolithography method after providing the separation layer over an entire surface of the substrate 1901.

The insulating film 1902 and the insulating film 1904 are formed using an insulating material such as silicon oxide, silicon nitride, silicon oxynitride, or silicon nitride oxide, by a CVD method, a sputtering method, or the like. For example, when the insulating film 1902 and the insulating film 1904 have a two-layer structure, preferably a silicon nitride oxide film is formed as a first insulating film and a silicon oxynitride film is formed as a second insulating film. Alternatively, a silicon nitride film may be formed as a first insulating film and a silicon oxide film may be formed as a second insulating film. The insulating film 1902 serves as a blocking layer which prevents an impurity element from the substrate 1901 from being mixed into the separation layer 1903 or an element formed thereover. The insulating film 1904 serves as a blocking layer which prevents an impurity element from the substrate 1901 or the separation layer 1903 from being mixed into an element formed thereover. By forming the insulating films 1902 and 1904 which serve as blocking layers in this manner, an element formed thereover can be prevented from being adversely affected by an alkali metal such as Na or an alkali earth metal from the substrate 1901, or an impurity element included in the separation layer 1903. Note that when quartz is used as the substrate 1901, the insulating films 1902 and 1904 may be omitted from the structure.

As the separation layer 1903, a metal film, a stacked-layer structure including a metal film and a metal oxide film, or the like can be used. As the metal film, a single-layer structure or a stacked-layer structure is formed using a film formed of any of the elements tungsten (W), molybdenum (Mo), titanium (Ti), tantalum (Ta), niobium (Nb), nickel (Ni), cobalt (Co), zirconium (Zr), zinc (Zn), ruthenium (Ru), rhodium (Rh), palladium (Pd), osmium (Os), iridium (Ir), and silicon (Si) or of an alloy material or a compound material containing such an element as a main constituent. These materials can be formed by use of a sputtering method, various CVD methods, such as a plasma CVD method, or the like. As the stacked-layer structure including a metal film and a metal oxide film, after the aforementioned metal film is formed, plasma treatment in an oxygen atmosphere or an N₂O atmosphere, or heat treatment in an oxygen atmosphere or an N₂O atmosphere is performed, so that oxide or oxynitride of the metal film can be formed on a surface of the metal film. For example, when a tungsten film is formed as the metal film by a sputtering method, a CVD method, or the like, plasma treatment is performed on the tungsten film so that a metal oxide film formed of tungsten oxide can be formed on a surface of the tungsten film. In this case, oxide of tungsten is expressed as WOₓ, where x is 2 to 3, and there are cases where x is 2 (WO₂), cases where x is 2.5 (W₂O₅), cases where x is 2.75 (W₄O₁₁), cases where x is 3 (WO₃), and the like. When forming the oxide of tungsten, there is no particular limitation on the value of x, and which oxide is to be formed may be determined in accordance with an etching rate or the like. Alternatively, for example, after a metal film (e.g., tungsten) is formed, an insulating film such as silicon oxide may be provided over the metal film by a sputtering method, and metal oxide may also be formed over the metal film (e.g., tungsten oxide over tungsten). In addition, as plasma treatment, the above high-density plasma treatment may also be performed, for example. Further, besides the metal oxide film, metal nitride or metal oxynitride may also be used. In such a case, plasma treatment or heat treatment under a nitrogen atmosphere or an atmosphere of nitrogen and oxygen may be performed on the metal film.

The semiconductor film 1905 is formed with a thickness of 10 to 200 nm (preferably, 30 to 150 nm) by a sputtering method, an LPCVD method, a plasma CVD method, or the like.

Next, as shown in FIG. 6B, the semiconductor film 1905 is crystallized by being irradiated with a laser beam. The semiconductor film 1905 may be crystallized by a method which combines laser beam irradiation with a thermal crystallization method which employs RTA or an annealing furnace or a thermal crystallization method which employs a metal element for promoting crystallization, or the like. Subsequently, the obtained crystalline semiconductor film is etched into a desired shape to form crystallized crystalline semiconductor films 1905a to 1905f, and a gate insulating film 1906 is formed so as to cover the crystalline semiconductor films 1905a to 1905f.

Note that the gate insulating film 1906 is formed using an insulating material such as silicon oxide, silicon nitride, silicon oxynitride, or silicon nitride oxide, by a CVD method, a sputtering method, or the like. For example, when the gate insulating film 1906 has a two-layer structure, preferably a silicon oxynitride film is formed as a first insulating film and a silicon nitride oxide film is formed as a second insulating film. Alternatively, a silicon oxide film may be formed as the first insulating film and a silicon nitride film may be formed as the second insulating film.

An example of a step for manufacturing the crystalline semiconductor films 1905a to 1905f will be briefly described hereinafter. A semiconductor layer having an amorphous structure is formed by a known method (a sputtering method, an LPCVD method, a plasma CVD method, or the like) and then crystallized by known crystallization treatment (laser crystallization, thermal crystallization, thermal crystallization using a catalyst such as nickel, or the like) so that a crystalline semiconductor layer is obtained, and the crystalline semiconductor layer is patterned into a desired shape after a resist mask is formed using a photomask so that the crystalline semiconductor films 1905a to 1905f are formed.

Note that as a laser oscillator for crystallization, a continuous wave laser beam (a CW laser beam) or a pulsed wave laser beam (a pulsed laser beam) can be used. As a laser beam which can be used here, a laser beam emitted from one or more of the following can be used: a gas laser, such as an Ar laser, a Kr laser, or an excimer laser; a laser whose medium is single crystalline YAG, YVO₄, forsterite (Mg₂SiO₄), YAlO₃, or GdVO₄, to which one or more of Nd, Yb, Cr, Ti, Ho, Er, Tm, and Ta has been added as a dopant; or polycrystalline (ceramic) YAQ Y₂O₃, YVO₄, YAlO₃, or GdVO₄, to which one or more of Nd, Yb, Cr, Ti, Ho, Er, Tm, and Ta has been added as a dopant; a glass laser; a ruby laser; an alexandrite laser; a Ti:sapphire laser; a copper vapor laser; or a gold vapor laser. Crystals with a large grain size can be obtained by irradiation with fundamental waves of such laser beams or second to fourth harmonics of the fundamental waves. For example, the second harmonic (532 nm) or the third harmonic (355 nm) of an Nd:YVO₄ laser (fundamental wave of 1064 nm) can be used. In this case, a power density of approximately 0.01 to 100 MW/cm² (preferably, 0.1 to 10 MW/cm²) is necessary. Irradiation is conducted with a scanning rate of approximately 10 to 2000 cm/sec. Note that a laser using, as a medium, single crystalline YAG YVO₄, forsterite (Mg₂SiO₄), YAlO₃, or GdVO₄, to which one or more of Nd, Yb, Cr, Ti, Ho, Er, Tm, and Ta has been added as a dopant, or polycrystalline (ceramic) YAG Y₂O₃, YVO₄, YAlO₃, or GdVO₄, to which one or more of Nd, Yb, Cr, Ti, Ho, Er, Tm, and Ta has been added as a dopant; an Ar ion laser; or a Ti:sapphire laser, can be continuously oscillated. Furthermore, pulse oscillation thereof can be performed at a repetition rate of 10 MHz or more by performing Q-switching operation, mode locking, or the like. When a laser beam is oscillated at a repetition rate of 10 MHz or more, during the time in which a semiconductor film is melted by the laser beam and then solidifies, the semiconductor film is irradiated with a next pulse. Accordingly, unlike in a case of using a pulsed laser with a low repetition rate, a solid-liquid interface can be continuously moved in the semiconductor film; therefore, crystal grains which have grown continuously in a scanning direction can be obtained.

Alternatively, as the crystallization treatment of a semiconductor layer having an amorphous structure, a sequential lateral solidification method (SLS method) may be used. In an SLS method, a sample is irradiated with a pulsed excimer laser beam through a slit-shaped mask. This is a method for continuously forming a crystal by the artificially controlled super-lateral growth and can be conducted by performing crystallization displacing a relative position of the sample and the laser beam every shot by an approximately the same length to that of the crystal which is super-laterally grown.

Further, the above-described high-density plasma treatment may be performed on the crystalline semiconductor films 1905a to 1905f to oxidize or nitride surfaces thereof, to form the gate insulating film 1906. For example, the gate insulating film 1906 is formed by plasma treatment in which a mixed gas which contains a rare gas such as He, Ar, Kr, or Xe, and oxygen, nitrogen dioxide, ammonia, nitrogen, hydrogen, or the like, is introduced. When excitation of the plasma in this case is performed by introduction of a microwave, high density plasma can be generated at a low electron temperature. The surface of the semiconductor film can be oxidized or nitrided by oxygen radicals (OH radicals may be included) or nitrogen radicals (NH radicals may be included) generated by this high-density plasma.

By treatment using such high-density plasma, an insulating film with a thickness of 1 to 20 nm, typically 5 to 10 nm, is formed over the semiconductor film. Because the reaction in this case is a solid-phase reaction, interface state density between the insulating film and the semiconductor film can be made very low. Because such high-density plasma treatment oxidizes (or nitrides) a semiconductor film (crystalline silicon, or polycrystalline silicon) directly, the insulating film can be formed with very little unevenness in its thickness. In addition, since crystal grain boundaries of crystalline silicon are also not strongly oxidized, very favorable conditions result. That is, by the solid-phase oxidation of the surface of the semiconductor film by the high-density plasma treatment shown here, an insulating film with good uniformity and low interface state density can be formed without excessive oxidation at crystal grain boundaries.

Note that as the gate insulating film 1906, just an insulating film formed by the high-density plasma treatment may be used, or an insulating film of silicon oxide, silicon oxynitride, silicon nitride, or the like may be formed thereover by a CVD method which employs plasma or a thermal reaction, to make stacked layers. In any case, when transistors include an insulating film formed by high-density plasma in a part of a gate insulating film or in the whole of a gate insulating film, unevenness in characteristics can be reduced.

Furthermore, in the crystalline semiconductor films 1905a to 1905f which are obtained by crystallizing a semiconductor film by irradiation with a continuous wave laser beam or a laser beam oscillated at a repetition rate of 10 MHz or more which is scanned in one direction, crystals grow in the scanning direction of the beam. When transistors are arranged so that the scanning direction is aligned with the channel length direction (the direction in which a carrier flows when a channel formation region is formed) and the above-described gate insulating layer is used in combination with the transistors, thin film transistors (TFTs) with less variation in characteristics and high electron field-effect mobility can be obtained.

Next, a first conductive film and a second conductive film are stacked over the gate insulating film 1906. Here, the first conductive film is formed with a thickness of 20 to 100 nm using a CVD method, a sputtering method, or the like. The second conductive film is formed with a thickness of 100 to 400 nm. The first conductive film and the second conductive film are formed using an element such as tantalum (Ta), tungsten (W), titanium (Ti), molybdenum (Mo), aluminum (Al), copper (Cu), chromium (Cr), or niobium (Nb), or using an alloy material or a compound material containing such an element as a main constituent. Alternatively, they are formed using a semiconductor material typified by polycrystalline silicon doped with an impurity element such as phosphorus. As examples of a combination of the first conductive film and the second conductive film, a tantalum nitride film and a tungsten film, a tungsten nitride film and a tungsten film, a molybdenum nitride film and a molybdenum film, and the like can be given. Because tungsten and tantalum nitride have high heat resistance, heat treatment for thermal activation can be performed after the first conductive film and the second conductive film are formed. In addition, in the case of using a three-layer structure instead of a two-layer structure, a stacked-layer structure including a molybdenum film, an aluminum film, and a molybdenum film may be used.

Next, a resist mask is formed using a photolithography method, and etching treatment for forming a gate electrode and a gate line is conducted, forming gate electrodes 1907 over the crystalline semiconductor films 1905a to 1905f. Here, an example in which the gate electrodes 1907 have a stacked-layer structure which includes a first conductive film 1907a and a second conductive film 1907b is described.

Next, as shown in FIG. 6C, the gate electrodes 1907 are used as masks, and an impurity element which imparts n-type conductivity is added to the crystalline semiconductor films 1905a to 1905f at a low concentration by an ion doping method or an ion implantation method. Subsequently, a resist mask is selectively formed by a photolithography method, and an impurity element which imparts p-type conductivity is added at a high concentration to the crystalline semiconductor films 1905a to 1905f. As an impurity element which imparts n-type conductivity, phosphorus (P), arsenic (As), or the like can be used. As an impurity element which imparts p-type conductivity, boron (B), aluminum (Al), gallium (Ga), or the like can be used. Here, phosphorus (P) is used as an impurity element which imparts n-type conductivity, and is selectively introduced into the crystalline semiconductor films 1905a to 1905f such that they contain phosphorus (P) at a concentration of 1×10¹⁵ to 1×10¹⁹/cm³. Thus, n-type impurity regions 1908 are formed. Further, boron (B) is used as an impurity element which imparts p-type conductivity, and is selectively introduced into the crystalline semiconductor films 1905c and 1905e such that they contain boron (B) at a concentration of 1×10¹⁹ to 1×10²⁰/cm³. Thus, p-type impurity regions 1909 are formed.

Next, an insulating film is formed so as to cover the gate insulating film 1906 and the gate electrodes 1907. The insulating film is formed as a single layer or stacked layers of a film containing an inorganic material such as silicon, oxide of silicon, or nitride of silicon, or a film containing an organic material such as an organic resin, by a plasma CVD method, a sputtering method, or the like. Next, the insulating film is selectively etched using anisotropic etching which etches mainly in a perpendicular direction, forming insulating films 1910 (also referred to as side walls) which are in contact with side surfaces of the gate electrodes 1907. The insulating films 1910 are used as masks for doping when LDD (lightly doped drain) regions are formed.

Next, using a resist mask formed by a photolithography method, the gate electrodes 1907, and the insulating films 1910 as masks, an impurity element which imparts n-type conductivity is added at a high concentration to the crystalline semiconductor films 1905a, 1905b, 1905d, and 1905f, to form n-type impurity regions 1911. Here, phosphorus (P) is used as an impurity element which imparts n-type conductivity, and it is selectively introduced into the crystalline semiconductor films 1905a, 1905b, 1905d, and 1905f such that they contain phosphorus (P) at a concentration of 1×10¹⁹ to 1×10²⁰/cm³. Thus, the n-type impurity regions 1911, which have a higher concentration than the impurity regions 1908, are formed.

By the above-described steps, n-channel thin film transistors 1900a, 1900b, 1900d, and 1900f and p-channel thin film transistors 1900c and 1900e are formed, as shown in FIG. 6D. Note that, here, a part of the charging circuit 108 that is connected to the battery 109 is shown by the n-channel thin film transistors 1900a and 1900f. A part of the transmitter/receiver circuit 107 is shown by the n-channel thin film transistors 1900b and 1900d and the p-channel thin film transistors 1900c and 1900e. Although not shown, the amplifier circuit 105 and the central arithmetic processing circuit 106 can be formed by use of the thin film transistors formed in the above step, as well.

Note that in the n-channel thin film transistor 1900a, a channel formation region is formed in a region of the crystalline semiconductor film 1905a which overlaps with the gate electrode 1907; the impurity regions 1911 which each form either a source region or a drain region are formed in regions which do not overlap with the gate electrode 1907 and the insulating films 1910; and low concentration impurity regions (LDD regions) are formed in regions which overlap with the insulating films 1910 and which are between the channel formation region and the impurity regions 1911. Further, the n-channel thin film transistors 1900b, 1900d, and 1900f are similarly provided with channel formation regions, low concentration impurity regions, and the impurity regions 1911.

Further, in the p-channel thin film transistor 1900c, a channel formation region is formed in a region of the crystalline semiconductor film 1905c which overlaps with the gate electrode 1907, and the impurity regions 1909 which each form either a source region or a drain region are formed in regions which do not overlap with the gate electrode 1907. Further, the p-channel thin film transistor 1900e is similarly provided with a channel formation region and the impurity regions 1909. Note that, here, the p-channel thin film transistors 1900c and 1900e are not provided with LDD regions; however, the p-channel thin film transistors may be provided with an LDD region, and the n-channel thin film transistor is not necessarily provided with an LDD region.

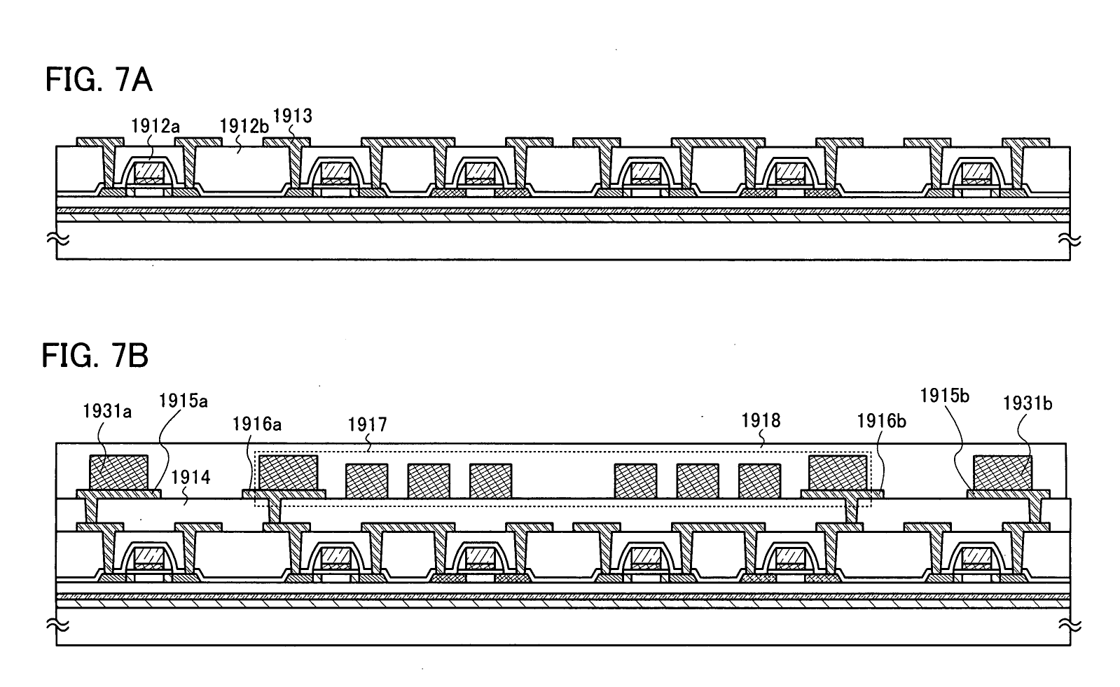

Next, as shown in FIG. 7A, an insulating film is formed as a single layer or stacked layers so as to cover the crystalline semiconductor films 1905a to 1905f, the gate electrodes 1907, and the like; and conductive films 1913, which are electrically connected to the impurity regions 1909 and 1911 which form the source regions or the drain regions of the thin film transistors 1900a to 1900f, are formed over the insulating film. The insulating film is formed as a single layer or stacked layers, using an inorganic material, such as oxide of silicon or nitride of silicon, an organic material, such as polyimide, polyamide, benzocyclobutene, acrylic, or epoxy, a siloxane material, or the like, by a CVD method, a sputtering method, an SOG method, a droplet discharge method, a screen printing method, or the like. Here, the insulating film has a two-layer structure. A silicon nitride oxide film is formed as a first insulating film 1912a, and a silicon oxynitride film is formed as a second insulating film 1912b. Further, the conductive films 1913 are formed as source electrodes and drain electrodes of the crystalline semiconductor films 1905a to 1905f.

Note that before the insulating films 1912a and 1912b are formed or after one or more thin films of the insulating films 1912a and 1912b are formed, heat treatment is preferably conducted for recovering the crystallinity of the semiconductor film, for activating an impurity element which has been added to the semiconductor film, or for hydrogenating the semiconductor film. As the heat treatment, thermal annealing, a laser annealing method, an RTA method, or the like is preferably used.

The conductive films 1913 are formed as a single layer or stacked layers, using any of the elements aluminum (Al), tungsten (W), titanium (Ti), tantalum (Ta), molybdenum (Mo), nickel (Ni), platinum (Pt), copper (Cu), gold (Au), silver (Ag), manganese (Mn), neodymium (Nd), carbon (C), and silicon (Si), or an alloy material or a compound material containing one of the above-mentioned elements as a main constituent, by a CVD method, a sputtering method, or the like. An alloy material containing aluminum as a main constituent corresponds to, for example, a material which contains aluminum as a main constituent and also contains nickel, or an alloy material which contains aluminum as a main constituent and which also contains nickel and one or both of carbon and silicon. The conductive films 1913 preferably employ, for example, a stacked-layer structure including a barrier film, an aluminum-silicon film, and a barrier film, or a stacked-layer structure including a barrier film, an aluminum-silicon film, a titanium nitride film, and a barrier film. Note that a barrier film corresponds to a thin film formed from titanium, nitride of titanium, molybdenum, or nitride of molybdenum. Aluminum and aluminum silicon, which have low resistance and are inexpensive, are ideal materials for forming the conductive films 1913. Further, generation of a hillock of aluminum or aluminum silicon can be prevented when upper and lower barrier layers are formed. Furthermore, when the barrier film is formed from titanium, which is a highly-reducible element, even if a thin natural oxide film is formed over the crystalline semiconductor film, the natural oxide film is chemically reduced, so good contact with the crystalline semiconductor film can be obtained.

Next, an insulating film 1914 is formed so as to cover the conductive films 1913, and over the insulating film 1914, conductive films 1915a and 1915b, which are each electrically connected to the conductive films 1913 which form source electrodes and drain electrodes of the crystalline semiconductor films 1905a and 1905f, are formed. Further, conductive films 1916a and 1916b, which are each electrically connected to the conductive films 1913 which form source electrodes and drain electrodes of the crystalline semiconductor films 1905b and 1905e, are formed. Note that the conductive films 1915a and 1915b may be formed of the same material at the same time as the conductive films 1916a and 1916b. The conductive films 1915a and 1915b and the conductive films 1916a and 1916b can be formed using any of the materials that the conductive films 1913 can be formed of, as mentioned above.

Next, as shown in FIG. 7B, a conductive film 1917 which serves as an antenna is formed so as to be electrically connected to the conductive films 1916a and 1916b. In addition, conductive films 1931a and 1931b which are electrically connected to the conductive films 1915a and 1915b, respectively, are formed at the same time as the conductive film 1917 which serves as an antenna is formed. Here, the conductive film 1917 which serves as an antenna corresponds to the antenna that is described in the above embodiment modes. Further, the thin film transistors 1900b to 1900e serve as the transmitter/receiver circuit which is described in the above embodiment modes. In addition, the conductive films 1931a and 1931b can function as a wiring which is electrically connected to a battery in a later step. Next, an insulating layer 1918 is formed to cover the conductive film 1917 and the conductive films 1931a and 1931b.

The conductive films 1917, 1931a, and 1931b are formed from a conductive material, using a CVD method, a sputtering method, a printing method, such as a screen printing method or a gravure printing method, a droplet discharge method, a dispensing method, a plating method, or the like. The conductive material is any of the elements aluminum (Al), titanium (Ti), silver (Ag), copper (Cu), gold (Au), platinum (Pt), nickel (Ni), palladium (Pd), tantalum (Ta), and molybdenum (Mo), or an alloy material or a compound material containing one of the above-mentioned elements as a main constituent, and has a single-layer structure or a stacked-layer structure.

For example, in the case of using a screen printing method to form the conductive film 1917 which serves as an antenna, the conductive film 1917 can be provided by selectively printing a conductive paste in which conductive particles having a grain size of several nm to several tens of μm are dissolved or dispersed in an organic resin. As conductive particles, metal particles of one or more of any of silver (Ag), gold (Au), copper (Cu), nickel (Ni), platinum (Pt), palladium (Pd), tantalum (Ta), molybdenum (Mo), titanium (Ti), and the like; fine particles of silver halide; or dispersive nanoparticles can be used. In addition, as the organic resin included in the conductive paste, one or more organic resins selected from among organic resins which serve as a binder, a solvent, a dispersing agent, or a coating material for the metal particles can be used. An organic resin such as an epoxy resin or a silicone resin can be given as representative examples. Further, when the conductive film is formed, it is preferable to conduct baking after the conductive paste is applied. For example, in the case of using fine particles containing silver as a main constituent (e.g., the grain size is greater than or equal to 1 nm and less than or equal to 100 nm) as a material for the conductive paste, the conductive film can be obtained by curing by baking at a temperature in the range of 150° C. to 300° C. Alternatively, fine particles containing solder or lead-free solder as a main constituent may be used. In that case, preferably fine particles having a grain size of 20 μm or less are used. Solder and lead-free solder have advantages such as low cost.

Further, although not shown, when the conductive film 1917 which serves as an antenna are formed, another conductive film may be separately formed such that it is electrically connected to the amplifier circuit 105, and that conductive film may be used as a wiring connected to the inner ear electrode 104.

Note that the insulating layer 1918 can be provided by a CVD method, a sputtering method, or the like as a single-layer structure or a stacked-layer structure which includes an insulating film containing oxygen and/or nitrogen, such as silicon oxide, silicon nitride, silicon oxynitride, or silicon nitride oxide; or a film containing carbon, such as DLC (diamond-like carbon); or an organic material, such as epoxy, polyimide, polyamide, polyvinyl phenol, benzocyclobutene, or acrylic; or a siloxane material, such as a siloxane resin.

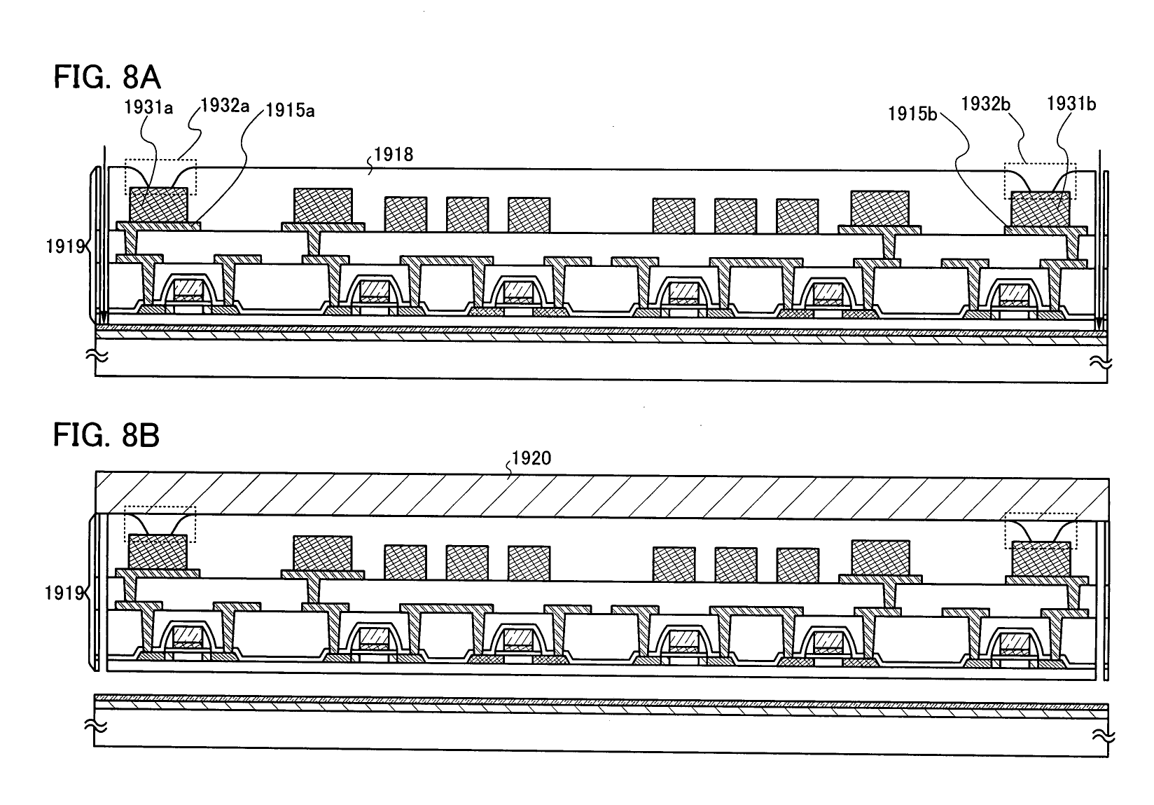

Next, as shown in FIG. 8A, openings 1932a and 1932b are formed in the insulating layer 1918 so that surfaces of the conductive films 1931a and 1931b are exposed.

Next, in this embodiment mode, openings are formed in a layer (hereinafter referred to as an “element formation layer 1919”) that includes the thin film transistors 1900a to 1900f, the conductive film 1917, the insulating layer 1918, and the like by laser beam irradiation.

Next, as shown in FIG. 8B, after an adhesive 1920 is attached to one surface (a surface where the insulating layer 1918 is exposed) of the element formation layer 1919, the element formation layer 1919 is separated from the substrate 1901. Here, after using laser beam (e.g., UV light) irradiation to form openings in regions where the thin film transistors 1900a to 1900f are not formed, the element formation layer 1919 can be separated from the substrate 1901 using a physical force. Alternatively, before the element formation layer 1919 is separated from the substrate 1901, an etchant may be introduced into the formed openings to selectively remove the separation layer 1903. As the etchant, a gas or liquid that contains halogen fluoride or a halogen compound is used. For example, chlorine trifluoride (ClF₃) is used as a gas that contains halogen fluoride. Accordingly, the element formation layer 1919 is separated from the substrate 1901. Note that a part of the separation layer 1903 may be left instead of it being removed entirely. By a part of the separation layer 1903 being left, consumption of the etchant and the amount of treatment time required for removing the separation layer can be reduced. Further, the element formation layer 1919 can be left over the substrate 1901 after the separation layer 1903 is removed. Furthermore, by the substrate 1901 being reused after the element formation layer 1919 is separated from it, cost can be reduced.

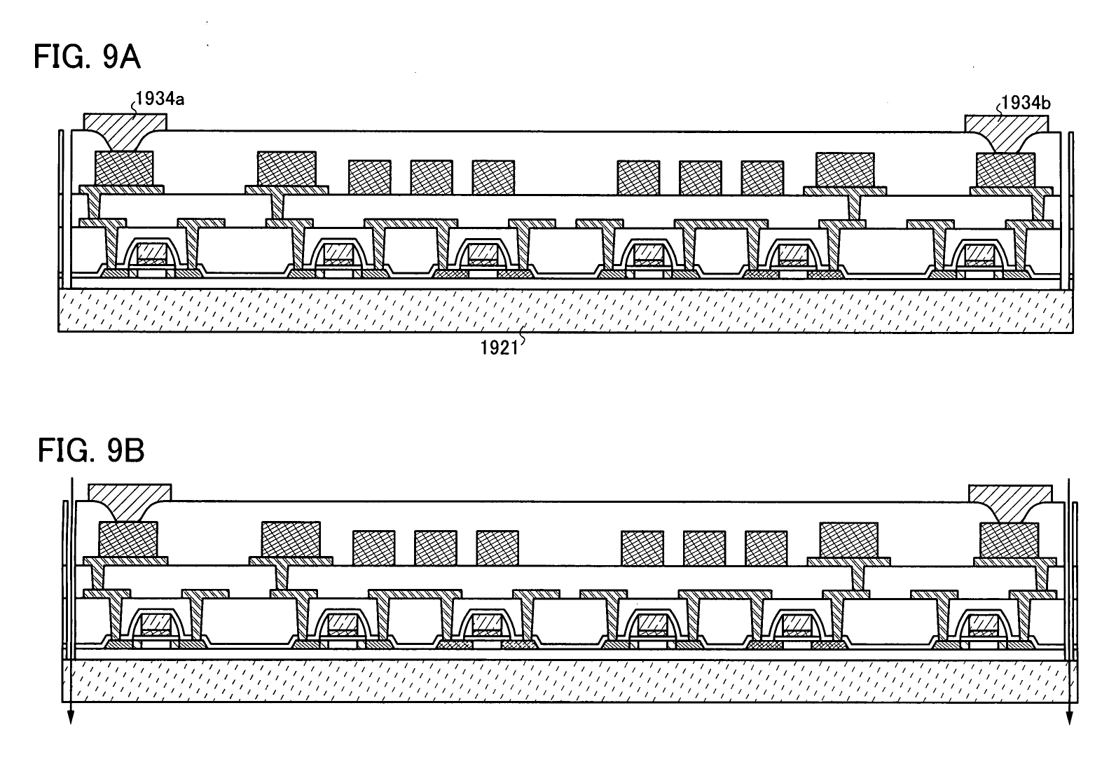

Next, as shown in FIG. 9A, a first housing 1921 is attached to the other surface (a surface where the insulating layer 1918 is exposed due to being separated from the substrate) of the element formation layer 1919. Then, the element formation layer 1919 is separated from the adhesive 1920. Consequently, here, a material having a low adhesive strength is used as the adhesive 1920. Next, conductive films 1934a and 1934b which are electrically connected to the conductive films 1931a and 1931b through the openings 1932a and 1932b respectively are formed selectively.

The conductive films 1934a and 1934b can be formed using a material and a manufacturing method which are similar to those used to form the conductive film 1917, as appropriate.

Note that, here, an example is shown in which the conductive films 1934a and 1934b are formed after the element formation layer 1919 is separated from the substrate 1901; however, the element formation layer 1919 may be separated from the substrate 1901 after the conductive films 1934a and 1934b are formed, as well.

The first housing 1921 is formed using a biologically inert material. Typically, a housing formed of a conductive material such as titanium, platinum, or gold or a housing formed of an insulating material such as an organic resin or a ceramic may be used. Furthermore, as the first housing 1921, a film formed using the above material may be used as well. When a film is used for the first housing 1921, the cochlear implant device 102, which is small and lightweight, is easily fitted to a body, and has little unevenness.

Next, as shown in FIG. 9B, in the case where a plurality of elements is formed over the substrate, the element formation layer 1919 is separated into separate elements. A laser irradiation apparatus, a dicing apparatus, a scribing apparatus, or the like can be used for the separation. Here, the plurality of elements formed over one substrate is separated from one another by laser light irradiation.

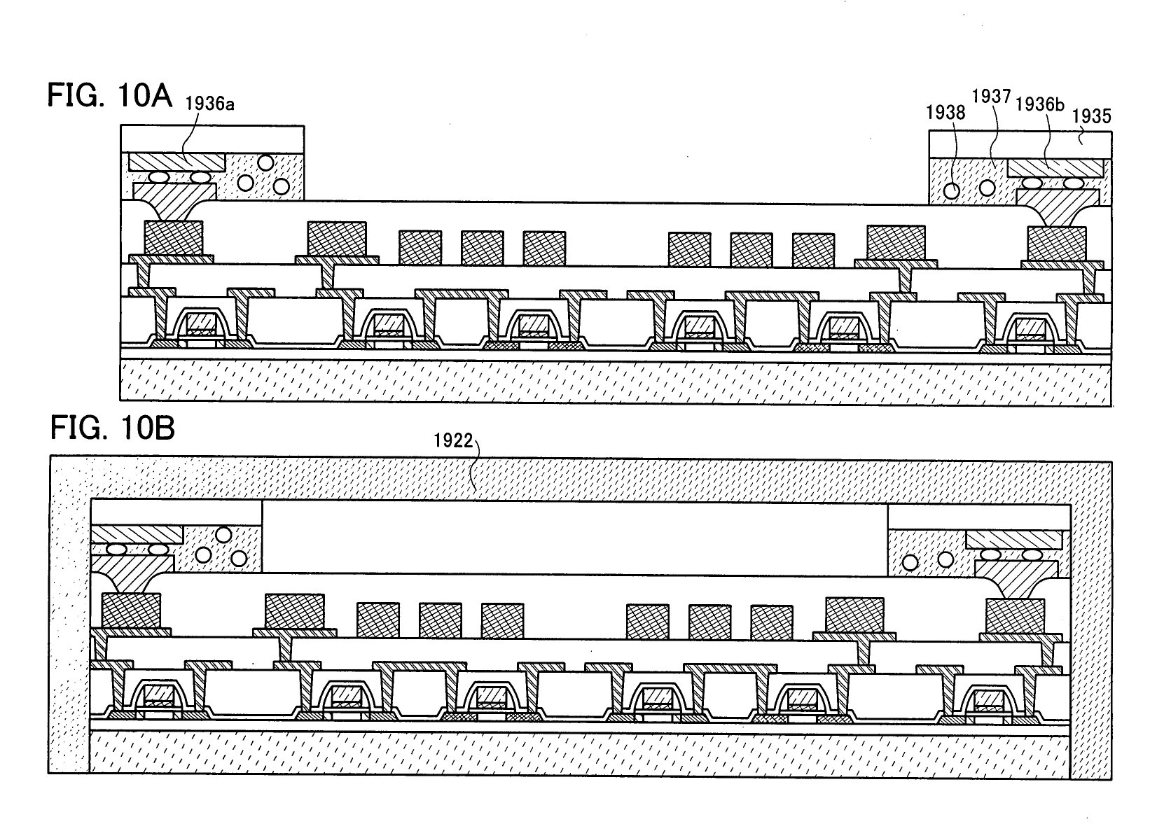

Next, as shown in FIG. 10A, the separated element is electrically connected to connecting terminals of the battery. Although not shown, the amplifier circuit 105 and the inner ear electrode 104 are electrically connected to each other. Here, an example is shown in which conductive films 1936a and 1936b which serve as connecting terminals of the battery, that are provided on a substrate 1935 are connected to the conductive films 1934a and 1934b, respectively, that are provided over the element formation layer 1919. Here, a case is shown in which the conductive film 1934a and the conductive film 1936a or the conductive film 1934b and the conductive film 1936b, are pressure-bonded to each other with a material that has an adhesive property such as an anisotropic conductive film (ACF) or an anisotropic conductive paste (ACP) interposed therebetween so that they are electrically connected to each other. Here, an example is shown in which conductive particles 1938 contained in a resin 1937 that has an adhesive property are used for connection. Alternatively, connection can be performed using a conductive adhesive agent such as a silver paste, a copper paste, or a carbon paste or using solder bonding or the like.

Next, as shown in FIG. 10B, a second housing 1922 is attached to the other surface (the surface where the insulating layer 1918 is exposed due to being separated from the substrate) of the element formation layer 1919 and the battery, followed by one or both of heat treatment and pressurization treatment for attachment of the first housing 1921 and the second housing 1922 to each other. The material given for the first housing 1921 can be used, as appropriate, for the second housing 1922. Note that when the first housing 1921 and the second housing 1922 are attached to each other, the inner ear electrode 104 is arranged so as to be protruded out from the housings. In addition, the first housing 1921 and the second housing 1922 may be attached to each other so that the space between the first housing 1921 and the second housing 1922 is drawn down to vacuum.

Furthermore, the surfaces of the first housing 1921 and the second housing 1922 are protected by a protective layer formed of silicon, fluorocarbon polymer, parylene, DLC, or the like, whereby the device is made safer for a body of a living thing.

As the first housing 1921 and the second housing 1922, materials (hereinafter referred to as antistatic materials) on which antistatic treatment for preventing static electricity or the like has been performed can be used. As a material that can prevent electrostatic charge, a metal, indium tin oxide (ITO), or a surfactant such as an amphoteric surfactant, a cationic surfactant, or a nonionic surfactant can be used. In addition to this, as an antistatic material, a resin material that contains a cross-linked copolymer having a carboxyl group and a quaternary ammonium base on its side chain or the like can be used. By attaching, mixing, or applying such a material to each of the housings, generation of static charge can be provided.

Note that the connection between the battery 109 and the charging circuit 108 and the connection between the inner ear electrode 104 and the amplifier circuit 105 may be made before the element formation layer 1919 is separated from the substrate 1901 (at a stage shown in FIG. 8A or FIG. 8B), or after the element formation layer 1919 is sealed with the first housing and the second housing (at a stage shown in FIG. 10B).

In a case where the battery is larger than the element, by forming a plurality of elements over one substrate, as shown in FIGS. 9A and 9B and FIGS. 10A and 10B, separating the elements, then connecting the elements to the battery, the number of elements which can be formed over one substrate can be increased. Accordingly, a cochlear implant device can be formed at low cost.

According to the above-described steps, a cochlear implant device can be manufactured. Note that in this embodiment, a step in which separation is performed after forming elements such as thin film transistors over the substrate has been described; however, the substrate over which elements are formed may be used as a product without performing separation. Further, when elements such as thin film transistors are provided over a glass substrate, and the glass substrate is then polished on the side opposite to the surface over which the elements are provided; or when a semiconductor substrate such as Si or the like is used and MOS transistors are formed, and the semiconductor substrate is then polished, thinning and miniaturization of a cochlear implant device can be achieved.

This application is based on Japanese Patent Application serial No. 2006-354767 filed with Japan Patent Office on Dec. 28, 2006, the entire contents of which are hereby incorporated by reference.

What is claimed is:

1. A cochlear implant system comprising:
a cochlear implant device; and
an extracorporeal sound collector operationally connected to the cochlear implant device,
wherein the cochlear implant device comprises an inner ear electrode, a first information processing circuit, a first transmitter/receiver circuit, a first charging circuit, and a first battery, and
wherein the extracorporeal sound collector comprises a microphone, an external input circuit, a second information processing circuit, a second transmitter/receiver circuit, a second charging circuit, and a second battery.

2. The cochlear implant system according to claim 1,
wherein the first transmitter/receiver circuit is electrically connected to the first information processing circuit and the first charging circuit,
wherein the first charging circuit is electrically connected to the first battery,
wherein the first battery is configured to supply power to the cochlear implant device,
wherein the second information processing circuit is electrically connected to the external input circuit and the second transmitter/receiver circuit, and the external input circuit is electrically connected to the microphone,
wherein the second transmitter/receiver circuit is electrically connected to the second charging circuit,
wherein the second charging circuit is electrically connected to the second battery, and
wherein the second battery is configured to supply power to the extracorporeal sound collector.

3. The cochlear implant system according to claim 1, wherein the second battery is charged from an external power source through the second charging circuit.

4. The cochlear implant system according to claim 1, wherein the first battery is charged through the first charging circuit with an electromagnetic wave received by the first transmitter/receiver circuit.

5. The cochlear implant system according to claim 1, wherein the microphone is a MEMS device.

6. A cochlear implant system comprising:
a cochlear implant device; and
an extracorporeal sound collector operationally connected to the cochlear implant device,
wherein the cochlear implant device comprises an inner ear electrode, a first amplifier circuit, a first central arithmetic processing circuit, a first transmitter/receiver circuit, a first charging circuit, and a first battery,
wherein the extracorporeal sound collector comprises a microphone, an external input circuit, a second amplifier circuit, a second central arithmetic processing circuit, a second transmitter/receiver circuit, a second charging circuit, and a second battery,
wherein the first transmitter/receiver circuit and the second transmitter/receiver circuit each comprise at least one antenna, a capacitor, a demodulation circuit, a decoding circuit, a logic operation/control circuit, a memory circuit, an encoding circuit, and a modulation circuit,
wherein the first charging circuit comprises a rectifier circuit configured to rectify an induced electromotive force generated in the antenna in the first transmitter/receiver circuit, a current/voltage control circuit, and a charge control circuit, and
wherein the second charging circuit comprises a rectifier circuit configured to rectify power inputted from an external power source, a current/voltage control circuit, and a charge control circuit.

7. The cochlear implant system according to claim 6,
wherein the first amplifier circuit is electrically connected to the inner ear electrode and the first central arithmetic processing circuit,
wherein the first transmitter/receiver circuit is electrically connected to the first central arithmetic processing circuit and the first charging circuit,
wherein the first charging circuit is electrically connected to the first battery,
wherein the first battery is configured to supply power to the cochlear implant device,
wherein the external input circuit is electrically connected to the microphone and the second amplifier circuit,
wherein the second amplifier circuit is electrically connected to the second central arithmetic processing circuit,
wherein the second transmitter/receiver circuit is electrically connected to the second central arithmetic processing circuit and the second charging circuit,
wherein the second charging circuit is electrically connected to the second battery, and
wherein the second battery is configured to supply power to the extracorporeal sound collector.

8. The cochlear implant system according to claim 6, wherein the second battery is charged from the external power source through the second charging circuit.

9. The cochlear implant system according to claim 6, wherein the first battery is charged through the first charging circuit with an electromagnetic wave which is received by the first transmitter/receiver circuit.

10. The cochlear implant system according to claim 6, wherein the microphone is a MEMS device.

11. A cochlear implant device comprising:
an inner ear electrode electrically connected to an information processing circuit;
a transmitter/receiver circuit electrically connected to the information processing circuit and a charging circuit; and
a battery electrically connected to the charging circuit.

12. The cochlear implant device according to claim 11, wherein the battery is configured to supply power to the cochlear implant device.

13. The cochlear implant device according to claim 11, wherein the battery is charged through the charging circuit with an electromagnetic wave which is received by the transmitter/receiver circuit.

14. A cochlear implant device comprising:
an amplifier circuit electrically connected to an inner ear electrode and a central arithmetic processing circuit;
a transmitter/receiver circuit electrically connected to a charging circuit and the central arithmetic processing circuit; and
a battery electrically connected to the charging circuit,
wherein the transmitter/receiver circuit comprises at least one antenna, a capacitor, a demodulation circuit, a decoding circuit, a logic operation/control circuit, a memory circuit, an encoding circuit, and a modulation circuit, and
wherein the charging circuit comprises a rectifier circuit configured to rectify an induced electromotive force generated in the antenna in the transmitter/receiver circuit, a current/voltage control circuit, and a charge control circuit.

15. The cochlear implant device according to claim 14, wherein the battery is configured to supply power to the cochlear implant device.

16. The cochlear implant device according to claim 14, wherein the battery is charged through the charging circuit with an electromagnetic wave which is received by the transmitter/receiver circuit.

17. An extracorporeal sound collector comprising:
a microphone electrically connected to an external input circuit;
an amplifier circuit electrically connected to the external input circuit and a central arithmetic processing circuit;
a transmitter/receiver circuit electrically connected to the central arithmetic processing circuit and a charging circuit; and
a battery electrically connected to the charging circuit,
wherein the transmitter/receiver circuit comprises at least one antenna, a capacitor, a demodulation circuit, a decoding circuit, a logic operation/control circuit, a memory circuit, an encoding circuit, and a modulation circuit, and
wherein the charging circuit comprises a rectifier circuit configured to rectify power inputted from an external power source, a current/voltage control circuit, and a charge control circuit.

18. The extracorporeal sound collector according to claim 17, wherein the battery is configured to supply power to the extracorporeal sound collector.

19. The extracorporeal sound collector according to claim 17, wherein the battery is charged through the charging circuit from the external power source.

20. The extracorporeal sound collector according to claim 17, wherein the microphone is a MEMS device.
